(19) United States
(12) Patent Application Publication
(10) Pub. No.: US 2020/0121704 A1
(43) Pub. Date: Apr. 23, 2020

(54) METHODS FOR TREATING HYPERTENSION AND ARTERIAL STIFFNESS

(71) Applicant: THE REGENTS OF THE UNIVERSITY OF COLORADO, A BODY CORPORATE, DENVER, CO (US)

(72) Inventors: CHRISTOPHER R. MARTENS, LOUISVILLE, CO (US); DOUGLAS R. SEALS, BOULDER, CO (US)

(21) Appl. No.: 16/477,639

(22) PCT Filed: Jan. 16, 2018
(86) PCT No.: PCT/US18/13886

§ 371 Date: Jul. 12, 2019

Related U.S. Application Data
(60) Provisional application No. 62/445,808, filed on Jan. 13, 2017.

(51) Int. Cl. A61K 31/706; A61P 9/12

(52) U.S. Cl. CPC A61K 31/706; A61P 9/12

(57) ABSTRACT

Methods of treating subjects suffering from elevated/stage 1 hypertension (formerly “pre-hypertension”) by administering to the subject a therapeutically effective amount of at least one nicotinamide adenine dinucleotide (NAD+) increasing compound, or functional analog, or prodrug, or salt thereof.

### CROSS REFERENCE TO RELATED APPLICATION

This application claims the benefit of priority under 35 U.S.C. § 119(e) to U.S. Provisional Patent Application Ser. No. 62/445,808, filed Jan. 13, 2017, which is incorporated herein by reference.

### STATEMENT OF FEDERALLY SPONSORED RESEARCH

This invention was made with government support under grant numbers T32 AG000279 and TR001082 awarded by the National Institutes of Health. The government has certain rights in the invention.

### TECHNICAL FIELD

This disclosure relates to methods of treating subjects suffering from pre-hypertension or hypertension, by administering to a subject in need of such treatment a therapeutically effective amount of at least nicotinamide adenine dinucleotide (NAD+) increasing compound, or salt thereof.

### BACKGROUND

Advancing age is the primary risk factor for the development of cardiovascular diseases (CVD), which remain the leading cause of death in developed societies (Mozaffarian, D. et al. Heart Disease and Stroke Statistics-2016 Update A Report From the American Heart Association. Circulation 133, E38-E360 (2016)). Aging increases the risk of CVD largely due to adverse changes to the vasculature including increases in blood pressure and stiffening of the large elastic arteries, both of which are recognized as major independent risk factors for age-associated CVD (Lakatta, E. G. & Levy, D. Arterial and cardiac aging: Major shareholders in cardiovascular disease enterprises Part I: Aging arteries: A “set up” for vascular disease. Circulation 107, 139-146 (2003)). As such, treatments aimed at lowering blood pressure and reducing large elastic artery stiffness hold promise for lowering the risk of developing CVD.

Modification of CVD risk factors through positive lifestyle behaviors such as healthy diet and regular aerobic exercise have, thus far, been the most effective strategies for reducing the risk of age-related CVD (LaRocca T J, Martens C R & Seals D R. Nutrition and other lifestyle influences on arterial aging. LID—S1568-1637(16)30227-6 [pii] LID—10.1016/j.arr.2016.09.002 [doi].—Ageing Res Rev. (2016). pii: S1568-1637(16)30227-6). In this regard, chronic calorie restriction (CR) has been shown to prevent or reverse age-related increases in CVD risk factors including increases in blood pressure and large elastic artery stiffening (Donato, A. J. et al. Life-long caloric restriction reduces oxidative stress and preserves nitric oxide bioavailability and function in arteries of old mice. Aging Cell 12, 772-783 (2013); Ahmet, I., et al. Cardioprotection by intermittent fasting in rats. Circulation 112, 3115-3121 (2005); Weiss, E. P. & Fontana, L. Caloric restriction: powerful protection for the aging heart and vasculature. Am. J. Physiol.-Heart Circul. Physiol. 301, H1205-H1219 (2011). But long-term adherence to CR and other healthy lifestyle behaviors remains low among adults in the United States and may lead to adverse health consequences in normal weight older adults who are at risk for malnutrition, loss of bone mineral density and sarcopenia (Villareal, D. T. et al. Effect of Two-Year Caloric Restriction on Bone Metabolism and Bone Mineral Density in Non-Obese Younger Adults: A Randomized Clinical Trial. J. Bone Miner. Res. 31, 40-51 (2016); Miller, S. L. & Wolfe, R. R. The danger of weight loss in the elderly. J Nutr Health Aging 12, 487-491 (2008)). As such, there is considerable interest in developing more practical alternative strategies, including compounds that mimic calorie restriction, for preventing or treating age-related CVD.

### SUMMARY

The present disclosure relates to a method of treating hypertension, pre-hypertension, and/or arterial stiffness in a subject in need of treatment thereof. The method involves the step of administering to the subject a therapeutically effective amount of at least one compound, wherein the at least one compound is at least nicotinamide adenine dinucleotide (NAD+) increasing compound. The at least nicotinamide adenine dinucleotide (NAD+) increasing compound may be nicotinamide riboside, a precursor of NAD+, a salt thereof, or a biologically active analog and/or prodrug thereof.

A subject receiving treatment for pre-hypertension (recently reclassified as either “elevated” [120-129 mmHg] or stage I hypertension [131-139 mmHg]) pursuant to the above-described method has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Optionally, these methods can further comprise administering to the subject a therapeutically effective amount of at least one anti-hypertensive compound with the at least one nicotinamide adenine dinucleotide (NAD+) increasing compound, or pharmaceutically acceptable salt thereof.

A subject receiving treatment for stage 2 or above hypertension pursuant to the above-described methods has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one nicotinamide adenine dinucleotide (NAD+) increasing compound, or pharmaceutically acceptable salt thereof.

The at least one nicotinamide adenine dinucleotide (NAD+) increasing compound may be administered to the subject to lower the systolic blood pressure, the diastolic blood pressure, the mean arterial pressure, or a combination of the systolic blood pressure and diastolic blood pressure of the subject. A subject receiving treatment pursuant to the above-described methods can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described methods may have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least one nicotinamide adenine dinucleotide (NAD+) increasing compound or pharmaceutically acceptable salt thereof.

This disclosure also provides methods of decreasing pre-hypertension blood pressure or elevated blood pressure in a subject, including administering to the subject a therapeutically effective amount of at least one nicotinamide adenine dinucleotide (NAD+) increasing compound. A subject being treated pursuant to this method can have a pre-hypertension blood pressure that comprises a systolic blood pressure in the range of 120 mmHg to 139 mmHg, a diastolic blood pressure in the range of 80 mmHg to 89 mmHg or a combination of a systolic blood pressure in the range of 120 mmHg to 139 mmHg and a diastolic blood pressure in the range of 80 mmHg to 89 mmHg. A subject being treated pursuant to this method can have an elevated blood pressure that comprises a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. For example, the subject may have an elevated blood pressure comprising a systolic blood pressure of at least 160 mmHg or a diastolic blood pressure of at least 95 mmHg. The administration of the at least one nicotinamide adenine dinucleotide (NAD+) increasing compound pursuant to this method can lower the systolic blood pressure, the diastolic blood pressure, the mean arterial pressure or a combination of the systolic blood pressure and diastolic blood pressure of the subject. Optionally, this method can further comprise administering to the subject a therapeutically effective amount of at least one anti-hypertensive compound with the at least one nicotinamide adenine dinucleotide (NAD+) increasing compound or pharmaceutically acceptable salt thereof.

This disclosure also provides methods of normalizing blood pressure in a subject having a history of pre-hypertension or hypertension. The method involves the step of administering to the subject a therapeutically effective amount of at least one nicotinamide adenine dinucleotide (NAD+) increasing compound, or a pharmaceutically acceptable salt thereof. The administration of the at least one compound pursuant to the above described method can normalize the systolic blood pressure, the diastolic blood pressure, the mean arterial pressure or a combination of the systolic blood pressure and diastolic blood pressure of the subject. A subject receiving treatment pursuant to the above-described method can have a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg. Alternatively, a subject receiving treatment pursuant to the above-described method can have a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg. Optionally, this method can further comprise administering to the subject a therapeutically effective amount of at least one anti-hypertensive compound with the at least nicotinamide adenine dinucleotide (NAD+) increasing compound or pharmaceutically acceptable salt thereof.

In these methods, the at least nicotinamide adenine dinucleotide (NAD+) increasing compound may be nicotinamide riboside, a precursor of NAD+, a salt thereof, such as the chloride salt thereof, or a biologically active analog and/or prodrug thereof.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF THE FIGURES

FIG. 1 shows human subject enrollment protocol for entry into a randomized, placebo-controlled crossover study testing the effects of nicotinamide riboside on systolic (SBP), diastolic (DBP) blood pressure and arterial stiffness in pre-hypertensive and normotensive individuals.

FIGS. 2A and 2B show the effect of nicotinamide riboside on resting systolic (FIG. 2A) and diastolic (FIG. 2B) blood pressures in all subjects compared with placebo.

FIGS. 3A-3F show the interaction of nicotinamide riboside administration and blood pressure status for systolic blood pressure (FIG. 3A and FIG. 3B) and diastolic pressure (FIG. 3C and FIG. 3D), and mean arterial pressure (FIG. 3E and FIG. 3F; P=0.08), compared to control.

FIG. 4A shows the effect of nicotinamide riboside administration on large elastic artery stiffness, as assessed by aortic pulse wave velocity (PWV), compared with placebo. FIG. 4B shows the near significant main effect of lowering PWV (P=0.07) of nicotinamide riboside administration alone, independent of blood pressure status.

FIG. 5 shows the effect of nicotinamide riboside on serum concentrations of the vasoconstrictor, endothelin-1 (ET-1).

### DETAILED DESCRIPTION

This disclosure provides methods for treating pre-hypertension, hypertension, and/or arterial stiffness in a subject in need of treatment thereof. In addition, the present disclosure also relates to methods of lowering blood pressure in a subject, methods of decreasing pre-hypertension blood pressure or elevated blood pressure in a subject, methods of normalizing blood pressure in a subject having a history of pre-hypertension or hypertension, and methods of reducing or reversing arterial stiffness in a subject. These methods will generally comprise administering to a subject in need of such therapy a therapeutically- or prophylactically-effective amount of at least one nicotinamide adenine dinucleotide (NAD+) increasing compound, or a biologically active analog and/or prodrug thereof, or salt thereof, to the subject.

### Definitions

The terms “administer”, “administering”, “administered” or “administration” refer to any manner of providing a drug (such as, a nicotinamide adenine dinucleotide (NAD+) increasing compound) to a subject or patient. Routes of administration can be accomplished through any means known by those skilled in the art. Such means include, but are not limited to, oral, buccal, intravenous, subcutaneous, intramuscular, by inhalation and the like.

As used herein, the term “antihypertensive compound or compounds” refers to one or more compounds that can reduce or lower blood pressure in a subject. Examples of antihypertensive compounds include, but are not limited to, diuretics, beta adrenergic blockers, calcium channel blockers, angiotensin converting enzyme inhibitors, vasodilators, sympatholytic drugs, and angiotensin II receptor antagonists.

As used herein, the phrase “diastolic blood pressure” refers to the minimum pressure exerted on the vessel walls when the heart muscle relaxes between beats and is filling with blood. Diastolic blood pressure is usually the second or bottom number in a blood pressure reading. Methods for measuring diastolic blood pressure are known to those skilled in the art.

As used herein, the term or phrase “hypertension” or “elevated blood pressure” refers to a systolic blood pressure in a subject of at least 140 mmHg, a diastolic blood pressure in a subject of at least 90 mmHg, a mean arterial pressure of at least 106 mmHg or a combination of a systolic blood pressure of at least 140 mmHg and a diastolic blood pressure of at least 90 mmHg in a subject. “Elevated blood pressure” refers to a systolic blood pressure in a subject between 120-129 mmHg, and a diastolic blood pressure of less than 80 mmHg.

Stage 1 high blood pressure (a diagnosis of hypertension) is now defined to be a systolic blood pressure between 130 and 139 mmHg or a diastolic blood pressure between 80 and 89 mmHg.

Stage 2 high blood pressure is now defined to be a systolic blood pressure over 140 mmHg or a diastolic blood pressure over 90 mmHg.

As used herein, the phrases “lowering blood pressure” or “lower blood pressure” refer to blood pressure in a subject that is reduced upon intake of a nicotinamide adenine dinucleotide (NAD+) increasing compound in accordance with the methods of the present disclosure. Any amount of blood pressure lowering is acceptable, as long as it is reduced by a statistically significant amount. Blood pressure is typically represented by systolic blood pressure and/or a diastolic blood pressure. Most frequently, blood pressure is represented as systolic blood pressure over diastolic blood pressure. Normal blood pressure in a human subject is a systolic blood pressure of below 120 mm Hg and a diastolic blood pressure of below 80 mm Hg (120/80 mm Hg) on average, but normal for a subject, such as a human being, can vary with the height, weight, fitness level, health, emotional state, age, etc., of a subject. The nicotinamide adenine dinucleotide (NAD+) increasing compounds of the present disclosure can be used to lower blood pressure, such as systolic blood pressure, diastolic blood pressure, mean arterial pressure or a combination of systolic blood pressure and diastolic blood pressure by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% over the initial or baseline blood pressure taken in a subject.

As used herein, the phrase “mean arterial blood pressure” “mean arterial pressure” or “MAP” refer to the product of cardiac output and peripheral vascular resistance. MAP is used to assess the hemodynamic status of a patient. More specifically, it is considered the perfusion pressure seen by organs in the body. Formulas for approximating MAP are well known to those skilled in the art. An example of a formula that can be used to calculate MAP is:

MAP=⅔ diastolic blood pressure+⅓ systolic blood pressure

As used herein, the term “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

Pre-hypertension is defined as being the top part of the range of normality of arterial hypertension, an individual lying within this range nonetheless being qualified as normotensive. Very recently, the American Heart Association and American College of Cardiology revised the current blood pressure guidelines such that the term formerly referred to has “pre-hypertension” now spans two distinct blood pressure categories: “elevated”, which refers to a systolic blood pressure between 120-129 mmHg and a diastolic blood pressure less than 80 mmHg; and “stage I hypertension”, which refers to a systolic blood pressure between 130-139 or a diastolic blood pressure between 80-89 mmHg. The terms “pre-hypertension” and “elevated/stage 1 hypertension” are used interchangeably throughout this disclosure. As used herein, the term “pre-hypertension” or “pre-hypertension blood pressure” or “stage 1 hypertension” refers to a systolic blood pressure in a subject in the range of 130 mmHg to 139 mmHg, a diastolic blood pressure in a subject in the range of 80 mmHg to 89 mmHg or a combination a systolic blood pressure in a subject in the range of 130 mmHg to 139 mmHg, a diastolic blood pressure in a subject in the range of 80 mmHg to 89 mmHg.

As used herein, the term “systolic blood pressure” refers to the peak pressure exerted on the walls of the arteries during the contraction phase of the ventricles of heart. Systolic blood pressure is usually the first or top number in a blood pressure reading. Methods for measuring systolic blood pressure are known to those skilled in the art.

As used herein, the term “subject” refers to an animal, preferably a mammal, including a human or non-human. The terms “patient” and “individual” and “subject” may be used interchangeably herein.

The terms “therapeutically effective amount” or “prophylactically effective amount” of a drug (namely, at least one nicotinamide adenine dinucleotide (NAD+) increasing compound, or a salt thereof) refers to a nontoxic but sufficient amount of the drug to provide the desired effect. The amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount.” However, an appropriate “therapeutically effective amount” or “prophylactically effective amount” in any individual case may be determined by one of ordinary skill in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term “nicotinamide adenine dinucleotide (NAD+) increasing compound” refers to any compound that increases nicotinamide adenine dinucleotide (NAD+) in serum or in a cell, such as, but not limited to, nicotinamide riboside. Examples of nicotinamide adenine dinucleotide (NAD+) increasing compounds include, but are not limited to, nicotinamide riboside, or a functional homolog or prodrug thereof, or a salt thereof. Some examples of nicotinamide riboside and its methods of manufacture are described in U.S. Pat. No. 8,106,184, which is incorporated herein in its entirety. The term “nicotinamide riboside” includes derivatives of nicotinamide riboside (e.g., nicotinamide riboside chloride). Nicotinamide riboside may be obtained from ChromaDex, Inc. (Irvine, Calif.). The term “nicotinamide adenine dinucleotide (NAD+) increasing compound” also includes functional metabolites, polymorphs, solvates and prodrugs of the nicotinamide riboside compounds described above.

As used herein, the term “prodrug” refers to a derivative of these nicotinamide riboside compounds that have chemically- or metabolically-cleavable groups and become by solvolysis or under physiological conditions compounds that are pharmaceutically active in vivo. Esters of carboxylic acids are an example of prodrugs that can be used in the dosage forms of the present disclosure. For example, methyl ester prodrugs may be prepared by reaction of a nicotinamide riboside compound in a medium such as methanol with an acid or base esterification catalyst (e. g., NaOH, H₂SO₄). Ethyl ester prodrugs are prepared in similar fashion using ethanol in place of methanol.

Preferred nicotinamide adenine dinucleotide (NAD+) increasing compound compounds are nicotinamide riboside and, especially, the chloride salt thereof.

### Methods of Treatment

As described above, this disclosure provides methods of treating pre-hypertension/elevated/stage 1 hypertension, hypertension, lowering blood pressure, normalizing blood pressure, and preventing, reducing, or reversing arterial stiffness in subjects in need of such treatment. The inventors have discovered that nicotinamide adenine dinucleotide (NAD+) increasing compounds can be used to treat pre-hypertension or hypertension, lower or normalize blood pressure and reduce arterial stiffness in these subjects.

The methods of this disclosure encompass establishing an initial or baseline blood pressure (such as a systolic blood pressure, a diastolic blood pressure, a mean arterial blood pressure or a combination of a systolic blood pressure and a diastolic blood pressure) for a subject. Methods for determining the blood pressure of a subject are well known in the art. For example, the systolic blood pressure and/or diastolic blood pressure of a subject can be determined using a sphygmomanometer (in mm of Hg) by a medical professional. Aneroid or electronic devices can also be used to determine the blood pressure of a subject and these devices and their use are also well known to those skilled in the art. Additionally, a 24-hour ambulatory blood pressure monitoring (hereinafter “ABPM”) device can be used to measure systolic blood pressure, diastolic blood pressure and heart rate. ABPM assesses systolic blood pressure, diastolic blood pressure and heart rate in predefined intervals (normally, the intervals are established at every 15 or 20 minutes, but any interval can be programmed) over a 24-hour period. The following parameters are then calculated from these readings after the data has been uploaded to a database. For example, ABPM can be used to measure the following: (1) the mean 24-hour systolic blood pressure of a subject; (2) the mean 24-hour diastolic blood pressure of a subject; (3) the mean daytime (the time period that constitutes “daytime” can readily be determined by those skilled in the art), systolic blood pressure of a subject; (4) the mean daytime diastolic blood pressure of a subject; (4) the mean nighttime (similarly, the time period that constitutes “nighttime” can readily be determined by those skilled in the art) systolic blood pressure of a subject; (5) the mean nighttime diastolic blood pressure of a subject; (6) the mean trough (the term “trough” refers to the time period at the end of the dosing period or the lowest point in drug levels and can readily be determined by those skilled in the art) systolic blood pressure of a subject; (7) the mean trough diastolic blood pressure of a subject; (8) the rate-pressure product (which is the product of heart rate and systolic blood pressure); and (9) the mean 24-hour mean rate-pressure product of a subject. The mean arterial pressure of a subject can be determined using a simple mathematical formula, such as the formula described previously herein (although alternative formulas are also known to those skilled in the art) once the systolic blood pressure and diastolic blood pressure of the subject has been determined. The time at which the blood pressure of the subject is determined is not critical for establishing the initial or baseline blood pressure reading. Once the initial or baseline blood pressure reading has been determined, a further determination is made by those skilled in the art as to whether or not the subject is suffering from (a) pre-hypertension or a pre-hypertension blood pressure; or (b) hypertension or an elevated blood pressure. For example, a baseline ABPM can be established 24-hours prior to beginning treatment of a subject to establish the initial or baseline ABPM in said subject. This initial or baseline APBM can also be used to determine whether the subject is suffering from pre-hypertension or hypertension.

Once a subject has been determined to be suffering from pre-hypertension (or pre-hypertension blood pressure) or hypertension (or elevated blood pressure), or if a subject has a history of suffering from pre-hypertension (or pre-hypertension blood pressure) or hypertension (or elevated blood pressure), the subject can be administered a therapeutically effective amount of at least one nicotinamide adenine dinucleotide (NAD+) increasing compound of this disclosure, thereby treating the subject. Preferably, the subject ingests the at least one nicotinamide adenine dinucleotide (NAD+) increasing compound on a daily basis.

The nicotinamide adenine dinucleotide (NAD+) increasing compound may be ingested one time per day, two times per day, three times per day (e.g., around each meal), four times per day, or more during the treatment period. For example, if the nicotinamide adenine dinucleotide (NAD+) increasing compound is nicotinamide riboside, the daily dosage of nicotinamide riboside may be greater than 100 mg, preferably greater than 250 mg, more preferably greater than 300 mg, most preferably greater than 400 mg or 500 mg or 1000 mg. The weekly dosage may be greater than 2100 mg/week, 2800 mg/week, 3500 mg/week, 4000 mg/week, 5000 mg/week, 6000 mg/week, 7000 mg/week, 8000 mg/week, or more. The daily dosage may be provided in a single unit dosage form (e.g., a single pill, capsule or tablet) or may be provided in smaller unit dosage forms if the oral composition is intended to be taken more than once per day.

The treatment period is ideally of sufficient time for the nicotinamide adenine dinucleotide (NAD+) increasing compound, such as nicotinamide riboside, to provide an improvement in the pre-hypertension, hypertension, blood pressure, and/or arterial stiffness. The treatment period may be at least 2 weeks, preferably at least 4 weeks, more preferably at least 6 weeks, or 8 weeks, or 10 weeks, or even indefinitely. In some instances, the treatment period will extend over multiple months (e.g., 1, 2, 3, 4, 5, 6, 7, 8, 9, 10, 11, 12 or more months) or multiple years (e.g., 1, 2, 3, 4, 5 or more years). Notably, the inventors discovered that within 6 weeks or less, twice daily administration of 500 mg of nicotinamide riboside to human subjects often results in significant improvements in pre-hypertension, hypertension, and arterial stiffness.

After the subject has ingested the at least one nicotinamide adenine dinucleotide (NAD+) increasing compound for a specified period of time (such as a day, a week, two weeks, three weeks, four weeks, etc.), a second blood pressure reading may be taken. This second blood pressure reading is compared to the initial or baseline blood pressure reading to determine whether there or not the subject exhibits a lower blood pressure (such as a lower systolic blood pressure, a lower diastolic blood pressure, a lower mean arterial pressure of a combination of a lower systolic blood pressure and a lower diastolic blood pressure). Any amount of statistically significant lower blood pressure (whether a statistically significant amount of a lower systolic blood pressure, a statistically significant amount of a lower diastolic blood pressure or a combination of a statistically significant amount of a lower systolic blood pressure and a lower diastolic blood pressure) is encompassed by the methods of the present disclosure. Moreover, the subject repeats the steps of ingesting the at least one nicotinamide adenine dinucleotide (NAD+) increasing compound (such as on a twice-daily basis), taking a subsequent blood pressure reading at a specified period of time and comparing the subsequent blood pressure reading to the initial or baseline blood pressure reading, until a desirable level of blood pressure reduction (or lower blood pressure) has been achieved in the subject. Such a desirable level of blood pressure reduction can be determined by those skilled in the art. Such a desirable level of blood pressure reduction includes, but is not limited to, the normalization of the subject's blood pressure to a systolic blood pressure of below 120 mm Hg, a diastolic blood pressure of 70 mm Hg or a combination of a systolic blood pressure of below 120 mm Hg and a diastolic blood pressure of 70 mmHg. Additionally, once the subject has obtained a desirable level of blood pressure reduction, the subject can continue to take the at least one nicotinamide adenine dinucleotide (NAD+) increasing compound indefinitely in order to maintain the desired level of blood pressure reduction.

Because the nicotinamide adenine dinucleotide (NAD+) increasing compounds of the present disclosure are effective in lowering blood pressure, these compounds can be used to treat subjects suffering from pre-hypertension (or pre-hypertensive blood pressure or elevated/stage 1 hypertension) or stage 2 hypertension. For example, the inventors discovered that in as little as six (4) weeks after beginning treatment with at least one nicotinamide adenine dinucleotide (NAD+) increasing compound, patients suffering from pre-hypertension or hypertension exhibited a lower blood pressure (i.e., a statistically significant lower systolic blood pressure, a statistically significant lower diastolic blood pressure, a statistically significant lower mean arterial pressure or a combination of a statistically significant lower systolic blood pressure and a statistically significant lower diastolic blood pressure). Moreover, it is also believed that the nicotinamide adenine dinucleotide (NAD+) increasing compounds of this disclosure can be used to further lower blood pressure in subjects already receiving one or more antihypertensive compounds. Thus, the nicotinamide adenine dinucleotide (NAD+) increasing compounds can be used as a monotherapy or as part of a combination therapy in lowering or decreasing blood pressure.

This disclosure also provides methods of reducing large artery stiffness in a subject. In these methods, the subject may have heart failure. These methods comprise administering to the subject a therapeutically effective amount of a nicotinamide adenine dinucleotide (NAD+) increasing compound, or a composition comprising the nicotinamide adenine dinucleotide (NAD+) increasing compounds, such as nicotinamide riboside, including as a chloride salt. These compounds or compositions comprising at least one nicotinamide adenine dinucleotide (NAD+) increasing compound may be administered in combination with at least one other agent useful for treating or reducing large artery stiffness. The at least one other agent may be selected from a diuretic, an angiotensin converting enzyme (ACE) inhibitor, an angiotensin II receptor blocker (ARB), a beta-blocker, a calcium-channel blocker, a statin, an organic nitrate, an organic nitrite, or combinations thereof. In these methods, measurements for determining artery stiffness may be acquired through any method known in the art, including, for example, carotid-femoral pulse wave velocity, an index of aortic stiffness, which is assessed using arterial tonometry or Doppler ultrasound, as would be understood by one skilled in the art.

Therapeutic Compositions

Compositions containing at least one nicotinamide adenine dinucleotide (NAD+) increasing compound in combination with at least one other pharmaceutical compound are contemplated for use in the methods of this disclosure. Using the excipients and dosage forms described below, formulations containing such combinations are a matter of choice for those skilled in the art. Further, those skilled in the art will recognize that various coatings or other separation techniques may be used in cases where the combination of compounds are incompatible.

The nicotinamide adenine dinucleotide (NAD+) increasing compounds of the present disclosure may also be present in the form of pharmaceutically acceptable salts. For use in medicine, the salts of the compounds of this disclosure refer to non-toxic “pharmaceutically acceptable salts” (see, International J. Pharm., 1986, 33, 201-217; J. Pharm. Sci., 1997 (January), 66, 1, 1). Other salts may, however, be useful in the preparation of nicotinamide adenine dinucleotide (NAD+) increasing compounds according to this disclosure or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one nicotinamide adenine dinucleotide (NAD+) increasing compounds or salts thereof, may be formulated in a variety of ways that is largely a matter of choice depending upon the delivery route desired. For example, solid dosage forms for oral administration include capsules, tablets, pills, powders and granules. In such solid dosage forms, these compounds may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

Liquid dosage forms for oral administration include pharmaceutically acceptable emulsions, solutions, suspensions, syrups and elixirs. In addition to the nicotinamide adenine dinucleotide (NAD+) increasing compounds, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., nicotinamide adenine dinucleotide (NAD+) increasing compounds or salts thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the nicotinamide adenine dinucleotide (NAD+) increasing compounds or salts thereof, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microeneapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present disclosure include powders, sprays, ointments and inhalants. The active compound(s) is mixed under sterile conditions with a pharmaceutically acceptable carrier and any needed preservatives, buffers or propellants which can be required. Opthalmic formulations, eye ointments, powders and solutions are also contemplated as being within the scope of this disclosure.

It will be understood that formulations used in accordance with the methods of the present disclosure generally will comprise a therapeutically effective amount of one or more nicotinamide adenine dinucleotide (NAD+) increasing compound. The phrase “therapeutically effective amount” or “prophylactically effective amount” as used herein means a sufficient amount of, for example, the composition, nicotinamide adenine dinucleotide (NAD+) increasing compound, or formulation necessary to treat the desired disorder, at a reasonable benefit/risk ratio applicable to any medical treatment. As with other pharmaceuticals, it will be understood that the total daily usage of a pharmaceutical composition of the disclosure will be decided by a patient's medical professional within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Formulations of the present disclosure are administered and dosed in accordance with sound medical practice, taking into account the clinical condition of the individual patient, the site and method of administration, scheduling of administration, and other factors known to medical practitioners.

Therapeutically effective or prophylactically effective amounts for purposes herein thus can readily be determined by such considerations as are known to those skilled in the art. The daily therapeutically effective or prophylactically effective amount of the nicotinamide adenine dinucleotide (NAD+) increasing compounds administered to a patient in single or divided doses range from about 0.01 to about 750 milligram per kilogram of body weight per day (mg/kg/day). More specifically, a patient may be administered from about 100 mg to about 2000 mg twice daily, preferably from about 250 mg to about 1000 mg twice daily and most preferably about 500 mg twice daily of nicotinamide riboside, as a chloride salt.

By way of example, and not of limitation, examples of the present invention will now be given.

### EXAMPLES

The inventors hypothesized that oral supplementation with the nicotinamide adenine dinucleotide (NAD+) increasing compound, nicotinamide riboside (NR) would reduce blood pressure (BP) and aortic pulse wave velocity (aPWV, a measure of large elastic artery stiffness). This hypothesis was tested in healthy middle-aged and older human adults (65±2 yrs, n=24) in a randomized, placebo-controlled crossover study.

Sixty subjects were consented for this study. This study was conducted in the Integrative Physiology of Aging Laboratory and the Clinical and Translational Research Center (CTRC) at the University of Colorado Boulder. All blood assays were performed at either Boulder Community Hospital or the University of Colorado Denver Anschutz Medical Campus CTRC Core Laboratory. All testing sessions were performed after an overnight fast and 24-hour abstention from exercise and alcohol consumption. All subjects refrained from all prescription medications for 24 hours and over the counter medications for 48 hours prior to all testing visits.

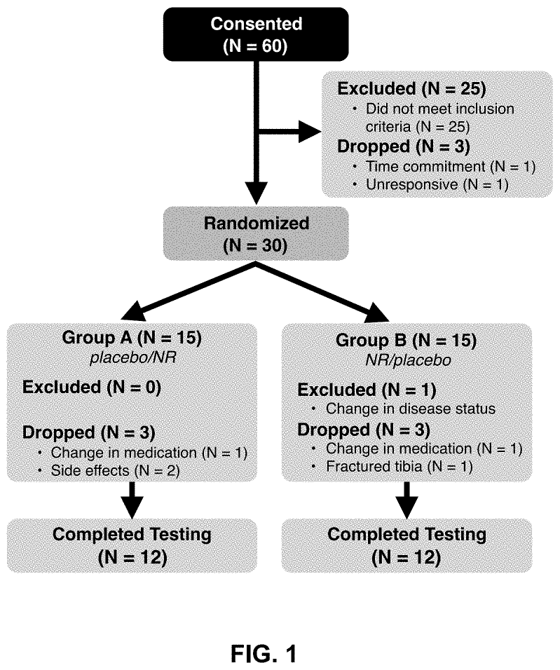

A randomized, double blinded, placebo controlled crossover design was implemented for this study. Healthy middle-aged and older (55-79 years) adult men and postmenopausal women were recruited from the Boulder/Denver metro area. Subjects were nonsmokers and free of clinical diseases as assessed by a medical history, physical examination, blood chemistry and resting and exercise ECG. Subjects were excluded if they had a BMI greater than 40 kg/m², had uncontrolled thyroid function, or had experienced a change in body mass (±2 kg) or medication status within the previous 3 months. Twenty-five subjects did not meet inclusion criteria and were excluded before randomization. Four subjects dropped out of the study prior to randomization due to time commitment and one subject was unresponsive to scheduling requests resulting in a total of 30 remaining subjects for randomization. Of these, fifteen subjects were randomized to Group A, which received placebo pills during the first 6 weeks of the study before crossing over to receive NR pills for 6 weeks. The remaining fifteen subjects were randomized to Group B, which received NR pills first followed by placebo. One subject was excluded from Group A due to a change in medication status that no longer met inclusion criteria and two subjects in Group A dropped out of the study due to a complaint of side effects. Two subjects were excluded from Group B due to a change in health or medication status that no longer met inclusion criteria and one subject dropped out of Group B due to a broken ankle, resulting in a total of 24 subjects who completed the trial (FIG. 1). These subjects were well matched by age, sex and baseline characteristics between groups and all baseline values were within normal clinical ranges, as shown in Table 1:

TABLE 1
Baseline subject characteristics

| | | | All Subjects |
|---|---|---|---|
| Subject Characteristic | Group A | Group B | Combined |
| Sex (M/F) | 5/7 | 6/6 | 11/13 |
| Age (years) | 64 ± 6 | 66 ± 9 | 65 ± 7 |
| Mass (kg) | 67 ± 16 | 69 ± 14 | 68 ± 15 |
| BMI (kg m⁻²) | 23 ± 4 | 24 ± 3 | 24 ± 4 |
| Total body fat (%) | 27 ± 10 | 29 ± 11 | 28 ± 10 |
| Systolic Blood Pressure (mmHg) | 122 ± 18 | 120 ± 17 | 121 ± 17 |
| Diastolic Blood Pressure (mmHg) | 77 ± 11 | 72 ± 9 | 74 ± 10 |
| Fasting Glucose (mg dL⁻¹) | 89 ± 8 | 87 ± 8 | 88 ± 8 |
| Total Cholesterol (mg dL⁻¹) | 192 ± 38 | 183 ± 36 | 187 ± 36 |
| HDL Cholesterol (mg dL⁻¹) | 69 ± 12 | 69 ± 25 | 69 ± 19 |
| LDL Cholesterol (mg dL⁻¹) | 107 ± 36 | 94 ± 23 | 101 ± 30 |

BMI, body mass index;
HDL, high-density lipoprotein;
LDL, low-density lipoprotein

No severe adverse events occurred during this study. NR was well tolerated at the dose tested, with all subjects consuming greater than 95% of NR pills administered. 14 treatment-emergent adverse events (AEs) were reported by 7 of the 30 participants enrolled in the study. All AEs were mild in severity and included symptoms of nausea, flushing, leg cramps and increased bruising during the NR condition and headache, skin rash, flushing, fainting and drowsiness during the placebo condition, as shown in Table 2.

TABLE 2
Treatment-emergent adverse events

| | Placebo # events | NR |
|---|---|---|
| Adverse Event (AE) | (# events/patient) | # events (# events/patient) |
| Headache | 4 (1) | 0 (0) |
| Nausea | 0 (0) | 1 (1) |
| Skin Rash | 1 (1) | 1 (1) |
| Flushing/Hot flashes | 2 (1) | 1 (1) |
| Fainting | 1 (1) | 0 (0) |
| Drowsiness | 1 (1) | 0 (0) |
| Leg cramps | 0 (0) | 1 (1) |
| Increased Bruising | 0 (0) | 1 (1) |

Data represent number (n) of times AE was reported.
Number of subjects reporting AEs (n = 7);
Number of subjects reporting ≥2 AE (n = 5).

Less than 1% of all subjects (N=2) dropped out of the study due to a complaint of side effects, both of which occurred while taking placebo (headache and skin rash). Considering that flushing was reported during both the placebo and NR conditions, it was determined that this side effect is unlikely associated with NR supplementation.

Clinical laboratory values were obtained from blood samples collected at the end of each intervention phase in 21 subjects. Complete blood work was not obtained in three subjects due to a failed catheterization during at least one of the testing phases. No meaningful differences were observed between treatment conditions for hematology (Table 3), blood chemistry (including markers of renal function and liver enzymes) (Table 4) or blood lipid profiles (Table 5). Although we observed a statistically significant difference in mean platelet volume (MPV) and the relative distribution of white blood cell types between conditions (Table 2), these changes were associated with extremely low effect sizes and were determined to be physiologically insignificant. Importantly, all clinical laboratory values remained within the normal reference range during both the placebo and NR conditions.

TABLE 3
Complete blood count

| | Placebo | NR | Reference Range |
|---|---|---|---|
| HGB (g/dL) | 13.8 ± 0.3 | 13.9 ± 0.2 | 12.6-16.3 g/dL |
| HCT (%) | 40.9 ± 0.7 | 41.1 ± 0.7 | 38.0-47.0% |
| WBC (10³/μL) | 4.5 ± 0.3 | 4.3 ± 0.3 | 3.8-9.5 10³/μL |
| RBC (10⁶/μL) | 4.5 ± 0.1 | 4.5 ± 0.1 | 4.18-5.33 10⁶/μL |
| RDW (%) | 12.9 ± 0.1 | 13.0 ± 0.2 | 11.5-15.2% |
| MCV (fL) | 92.1 ± 1.0 | 92.3 ± 0.9 | 81.5-99.8 fL |
| MCH (pg) | 31.1 ± 0.3 | 31.2 ± 0.3 | 27.9-34.1 pg |
| MCHC (g/dL) | 33.8 ± 0.2 | 33.8 ± 0.2 | 32.4-36.7 g/dL |
| MPV (fL) | 10.8 ± 0.2 | 10.7 ± 0.2* | 8.7-11.7 fL |
| platelet count | 198 ± 11 | 206 ± 14 | 150-400 10³/μL |
| (10³/μL) | | | |
| lymphocytes | | | |
| relative (%) | 29.5 ± 1.5 | 29.5 ± 1.7 | 15.0-45.0% |
| absolute (10³/μL) | 1.29 ± 0.08 | 1.18 ± 0.08 | 1.00-3.00 10³/μL |
| monocytes | | | |
| relative (%) | 10.2 ± 0.5 | 10.7 ± 0.6 | 4.5-13.0% |
| absolute (10³/μL) | 0.45 ± 0.03 | 0.52 ± 0.08 | 0.30-0.80 10³/μL |
| neutrophils | | | |
| relative (%) | 55.9 ± 1.3 | 55.3 ± 1.4 | 39.3-74.2% |
| absolute (10³/μL) | 2.52 ± 0.17 | 2.41 ± 0.19 | 1.70-6.50 10³/μL |
| eosinophils | | | |
| relative (%) | 3.5 ± 0.5 | 2.9 ± 0.5* | 0.6-7.6% |
| absolute (10³/μL) | 0.17 ± 0.03 | 0.14 ± 0.03 | 0.03-0.40 10³/μL |
| basophils | | | |
| relative (%) | 0.9 ± 0.1 | 1.3 ± 0.2* | 0.3-1.7% |
| absolute (10³/μL) | 0.04 ± 0.00 | 0.05 ± 0.01 | 0.02-0.10 10³/μL |

Data are mean ± standard error of mean (SEM);
HGB, hemoglobin;
HCT, hematocrit;
WBC, white blood count;
RBC, red blood count;
RDW, red cell distribution width;
MCV, mean corpuscular volume;
MCH, mean corpuscular hemoglobin;
MCHC, mean corpuscular hemoglobin concentration;
MPV, mean platelet volume.
*P < 0.05 vs. placebo.

TABLE 4
Comprehensive metabolic panel

| | Placebo | NR | Reference Range | |
|---|---|---|---|---|
| total protein (g/dL) | 6.3 ± 0.1 | 6.3 ± 0.1 | 6.3-8.2 | g/dL |
| albumin (g/dL) | 3.7 ± 0.1 | 3.6 ± 0.1 | 3.5-5.0 | g/dL |
| AST (SGOT) (IU/L) | 33.2 ± 2.7 | 32.1 ± 1.5 | 14-46 | IU/L |
| alk phosphatase (IU/L) | 63.6 ± 3.2 | 63.4 ± 2.9 | 38-126 | IU/L |
| total bilirubin (mg/dL) | 0.8 ± 0.1 | 0.8 ± 0.1 | 0.1-1.4 | mg/dL |
| ALT (SGPT) (IU/L) | 32.4 ± 1.6 | 30.8 ± 2.4 | 9-52 | IU/L |
| creatinine (mg/dL) | 0.9 ± 0.04 | 0.9 ± 0.03 | 0.6-1.0 | mg/dL |
| BUN (mg/dL) | 18 ± 1 | 17 ± 1 | 7-23 | mg/dL |
| eGFR (ml/min/1.73 m²) | 75 ± 3 | 74 ± 3 | ≥60 | mg/dL |
| calcium (mg/dL) | 9.2 ± 0.1 | 9.2 ± 0.1 | 8.5-10.4 | mg/dL |
| glucose (mg/dL) | 87 ± 1 | 87 ± 1 | 70-100 | mg/dL |
| sodium (mEq/L) | 139 ± 0 | 139 ± 1 | 134-144 | mEq/L |
| potassium (mEq/L) | 4.2 ± 0.1 | 4.2 ± 0.1 | 3.5-5.2 | mEq/L |
| chloride (mEq/L) | 104 ± 1 | 104 ± 1 | 97-110 | mEq/L |
| CO₂ (mEq/L) | 26 ± 1 | 26 ± 1 | 22-31 | mEq/L |

Data are mean ± standard error of mean (SEM);
AST, aspartate aminotransferase;
SGOT, serum-oxaloacetic transaminase;
alk, alkaline;
ALT, alanine aminotransferase;
SGPT, serum glutamic-pyruvic transaminase;
BUN, blood urea nitrogen;
eGFR, estimated glomerular filtration rate;
CO₂, carbon dioxide.

TABLE 5
Total lipid panel

| | Placebo | Niagen ™ | Reference Range |
|---|---|---|---|
| Total cholesterol (mg/dL) | 167 ± 5 | 171 ± 6 | 140-220 mg/dL |
| Triglycerides (mg/dL) | 90 ± 16 | 90 ± 14 | 35-135 mg/dL |
| HDL-C (mg/dL) | 60 ± 4 | 60 ± 5 | 40-85 mg/dL |
| LDL-C (mg/dL) | 90 ± 4 | 94 ± 5 | 80-100 mg/dL |
| VLDL-C (mg/dL) | 18 ± 3 | 17 ± 3 | 8-25 mg/dL |
| N-HDL-C (mg/dL) | 108 ± 6 | 111 ± 6 | 90-129 mg/dL |

Data are mean ± standard error of mean (SEM);
HDL-C, high-density lipoprotein cholesterol;
LDL-C, low-density lipoprotein cholesterol;
VLDL-C, very low-density lipoprotein cholesterol;
N-HDL-C, non-HDL cholesterol.

Statistical analyses were performed using the R statistical computing language (version 3.2.2). Prior to unblinding of the data, all continuous variables were assessed for normality using the Shapiro-Wilk test of normality with a p-value <0.01 used to identify non-normally distributed data. The normality of all data was confirmed by examining individual frequency histograms. Non-normally distributed data were either log-transformed prior to analysis or a non-parametric test was applied as appropriate. All variables were assessed for the presence of carryover effects using a linear regression with treatment order included as a factor. Repeated measures (e.g., body mass, blood pressure) were analyzed using a repeated measures analysis of variance (ANOVA) with time and treatment condition (NR vs. placebo) included as factors. All continuous variables collected at the end of each intervention phase were compared between phases using a paired t-test. If a continuous variable was non-normally distributed, and normality was not corrected by log-transformation, the effect of treatment condition was analyzed using a Wilcoxon signed rank test. Subsequent analyses were performed by blood pressure status using a 2×2 repeated measures ANOVA with between group (normotensive, pre-hypertensive) and within-group (placebo, NR) comparisons. When a significant group by condition interaction was revealed (P<0.05), the within group differences were assessed using a paired t-test. P-values less than 0.05 were considered statistically significant.

### NAD+ and Related Metabolites

Oral NR supplementation effectively elevated levels of NAD⁺ in PBMCs by about 60% compared with placebo (mean change=6.2 pMol/mg protein) (Table 6). The mean level of NADP⁺ also increased, but did not reach statistical significance. Of note, NR also elevated levels of nicotinic acid adenine dinucleotide (NAAD) nearly 5-fold above the placebo condition. NR also elevated the mean concentration of nicotinamide (NaM), but this was not statistically significant. An increase in NaM would suggest an increase in the activity of NAD⁺-consuming enzymes, which catalyze the breakdown of NAD⁺ into NaM and ADP-Ribose. Though not significant, we also observed an ˜1.5-fold increase in nicotinamide mononucleotide (NMN; mean change=0.72 pMol/mg protein), which may indicate the possible conversion of NR to NMN by nicotinamide riboside kinase (NRK) enzymes or further metabolism of NaM into NMN by nicotinamide phosphoribosyltransferase (NAMPT).

In addition to boosting NAD⁺-specific metabolites in PBMCs, we also observed increases in the mean concentration of other metabolites involved in the regulation of energy production and metabolism, including adenosine and adenosine triphosphate (ATP; mean change=699 pMol/mg protein) (Table X). NR supplementation also tended to raise levels of adenosine diphosphate (ADP) and adenosine monophosphate (AMP), though increases in these metabolites did not reach statistical significance (Table 6). Collectively, these findings indicate that chronic NR supplementation effectively stimulates NAD⁺ metabolism in healthy middle-aged and older men and women.

TABLE 6
NAD+ and Related Metabolites

| Metabolite (pMol/mg protein) | Placebo | NR |
|---|---|---|
| NaM | 277.4 ± 72.8 | 383.8 ± 282.7 |
| Adenosine | 10.1 ± 4.8 | 14.4 ± 9.7* |
| Uridine | 128.0 ± 60.2 | 278.3 ± 718.4 |
| NR | <LOQ | <LOQ |
| Cytidine | 2.9 ± 3.7 | 4.7 ± 6.6 |
| Guanosine | 1.5 ± 5.4 | 0.5 ± 1.5 |
| 2-3 cAMP | 0.0 ± 0.0 | 0.0 ± 0.0 |
| 3-5 cAMP | 0.0 ± 0.0 | 0.0 ± 0.0 |
| cCMP | 0.0 ± 0.0 | 1.8 ± 8.4 |
| NAD+ | 10.1 ± 6.7 | 16.2 ± 14.1* |
| NMN | 0.5 ± 1.3 | 1.2 ± 3.3 |
| cGMP | 0.0 ± 0.0 | 0.0 ± 0.0 |
| NAAD | 0.2 ± 0.6 | 1.4 ± 2.2* |
| FAD | 9.9 ± 4.6 | 10.2 ± 5.9 |
| CMP | 48.8 ± 25.1 | 137.2 ± 366.0 |
| AMP | 269.6 ± 132.8 | 770.8 ± 1884.3 |
| UMP | 551.2 ± 243.7 | 1113.7 ± 2374.2 |
| GMP | 513.9 ± 195.5 | 1010.1 ± 2114.8 |
| NADP | 7.7 ± 4.0 | 8.9 ± 8.7 |
| CDP | 34.8 ± 16.0 | 67.7 ± 113.1 |
| UDP | 151.2 ± 79.5 | 270.0 ± 420.7 |
| ADP | 566.2 ± 231.6 | 1133.8 ± 1880.4 |
| GDP | 115.3 ± 51.0 | 198.4 ± 329.0 |
| CTP | 22.6 ± 12.4 | 41.0 ± 68.4 |
| UTP | 276.1 ± 123.5 | 340.3 ± 150.1 |
| ATP | 1736.3 ± 861.7 | 2435.7 ± 1230.3* |
| GTP | 279.5 ± 114.2 | 327.1 ± 119.1 |

*p < 0.05.
Data are mean ± SD. N = 21.

Blood Pressure

Resting brachial artery blood pressure and heart rate were measured in the seated position after 5 minutes of rest in a quiet examination room using a semi-automated blood pressure device (DYNAMAP™ XL, Johnson & Johnson, Arlington, Tex., USA). Measurements were made multiple times from the non-dominant arm, with 2 minutes of quiet rest between recordings. Repeat measurements were made until 3 blood pressure values were obtained that were within 5 mmHg of one another. These values were then averaged to determine resting systolic and diastolic blood pressure, pulse pressure, mean arterial pressure, and resting heart rate. Baseline blood pressure values were obtained using the above-described protocol on two separate testing days prior to the initiation of the first intervention arm and were averaged to determine baseline blood pressure status (i.e., normotensive vs. pre-hypertensive) for subsequent analyses.

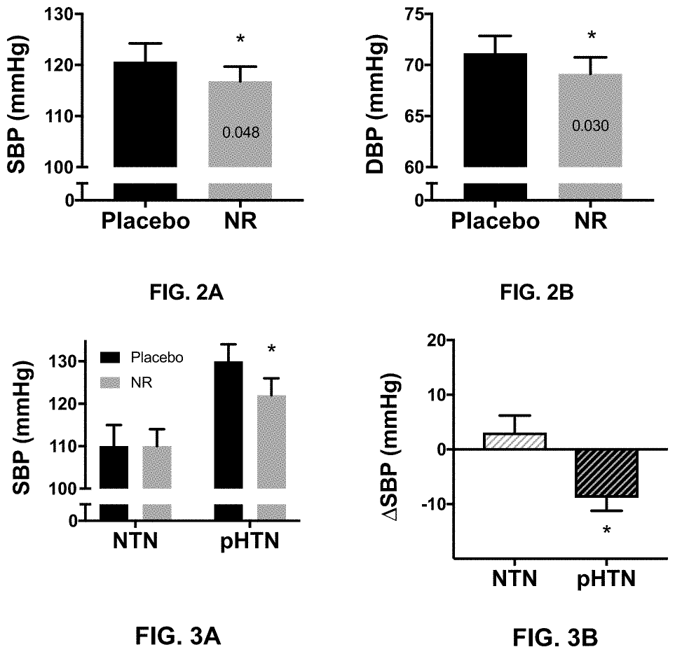

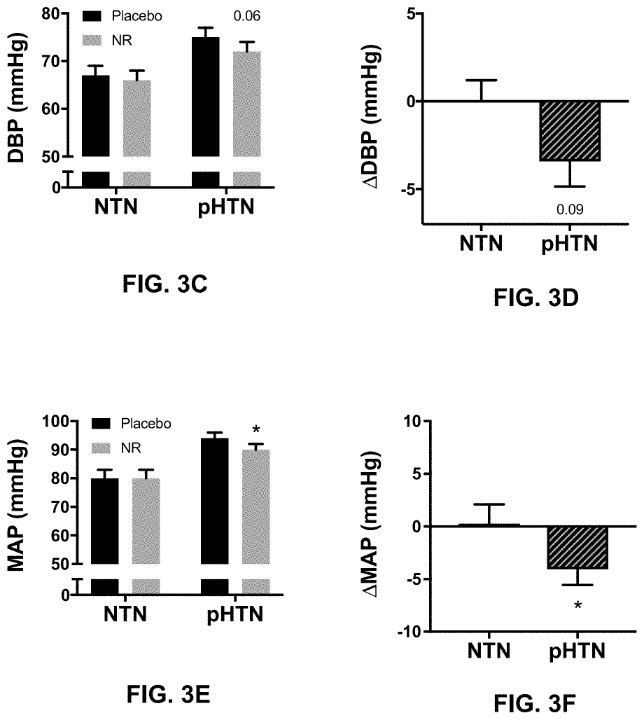

Resting systolic and diastolic blood pressures were significantly reduced (P<0.05) in all subjects during the NR condition compared with placebo (FIGS. 2A, 2B). To determine if this effect was greater in subjects with elevated baseline blood pressure, subjects were characterized as either normotensive (SBP <120 mmHg; DBP <80 mmHg) or pre-hypertensive (SBP 120-139 mmHg; DBP 80-89 mmHg) based on their average baseline blood pressure obtained on two separate days. The inventors observed a near significant interaction between treatment condition (NR vs. placebo) and blood pressure status for systolic blood pressure (FIG. 3A; P=0.07), with no effect on diastolic pressure (FIG. 3C), and a significant effect on mean arterial pressure (FIG. 3E; P=0.08). A post-hoc analysis exploring the effect of NR on the change in blood pressure between each blood pressure group, revealed a significant reduction in mean and systolic pressures in pre-hypertensive individuals (P<0.01), with NR lowering systolic blood pressure by 8 mmHg compared with placebo (FIG. 3B). Diastolic blood pressure also tended to be reduced in prehypertensive individuals; however this effect was lower in magnitude (3 mmHg reduction) and approached significance (FIG. 3D; P=0.09). The reduction in MAP was also significantly different in prehypertensive individuals compared with normotensive individuals (FIG. 3E; P=0.009) There was no effect of NR on blood pressure in normotensive individuals.

Arterial Stiffness

Carotid-femoral PWV, the gold-standard assessment of elastic artery stiffness in humans, was measured using transcutaneous tonometry. Pressure waveforms were recorded simultaneously from the carotid and femoral arteries using two identical non-invasive pressure tonometers (SPT-301, Millar Instruments, Houston, Tex., USA). The transit distance was calculated as the distance between the carotid and femoral recording sites, after subtracting the distance between the carotid recording site and the sternal notch. PWV was calculated as the transit distance divided by the time delay between the foot of each pressure wave.

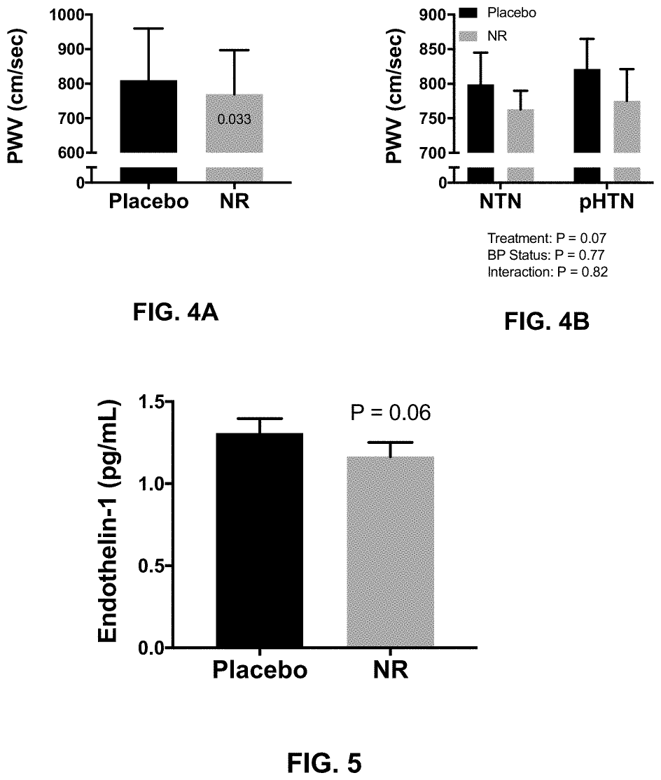

Large elastic artery stiffness, as assessed by aortic pulse wave velocity (PWV), was significantly reduced following NR supplementation compared with placebo (FIG. 4A). We also observed no interaction between PWV and blood pressure status (P=0.82), but did observe a near significant main effect (P=0.07) of treatment alone (FIG. 4B) further suggesting that NR lowered PWV independent of blood pressure status.

This is the first study to examine the safety and potential therapeutic benefit of chronic nicotinamide riboside supplementation on physiological function in humans. This study demonstrated that six weeks of oral supplementation with NR is safe and effective at reducing blood pressure and large elastic artery stiffness in healthy middle-aged and older adults. Because elevated blood pressure and arterial stiffness are important cardiovascular risk factors, these findings have important implications for the treatment and prevention of age-related CVD.

Endothelin-1

To gain potential insight into the underlying mechanisms by which nicotinamide riboside lowers blood pressure, we assessed plasma concentrations of endothelin-1 (ET-1), a potent vasoconstrictor that is released by the vascular endothelium to regulate vascular smooth muscle tone. Our findings suggest that compared with placebo, nicotinamide riboside supplementation lowers serum ET-1 concentrations. Thus, nicotinamide riboside may lower blood pressure and arterial stiffness by suppressing ET-1-mediated vasoconstriction.

NR Supplementation and Blood Pressure

Supplementation with NR moderately lowered systolic, diastolic and mean arterial blood pressure in all subjects; however, the greatest effect occurred in individuals with elevated baseline blood pressure within the pre-hypertensive range. This finding is important as approximately one third of blood pressure-attributed deaths occur in individuals with elevated blood pressure that is below the clinical cutoff for hypertension. Moreover, the magnitude by which NR lowered blood pressure in pre-hypertensive individuals is clinically meaningful, as even modest reductions in blood pressure have been shown to have a profound effect on lowering CVD risk. The mechanisms by which NR lowered blood pressure remain unclear but may be related to decreased oxidative stress via the activation of SIRT-1. Little is currently known about the relation between SIRT-1 and the regulation of blood pressure; however, activation of SIRT-1 with the pharmacological activator SRT1720, protects against the development of hypertension in Klotho deficient mice (a model of accelerated aging), by lowering vascular oxidative stress. This study is the first to demonstrate a reduction in blood pressure using an endogenous NAD+ precursor compound in humans.

NR Supplementation and Large Elastic Artery Stiffness

Aortic stiffness increases during normal aging and has been associated with elevated risk for cardiovascular events and CVD-related mortality. In the present study, the inventors found that oral supplementation with NR for 6 weeks reduced aortic stiffness in healthy middle-aged and older adults, providing the first translational evidence for the role of a NAD+ precursor in lowering arterial stiffness in humans. The exact cause of age-related arterial stiffening is not completely understood but is thought to be mediated by a combination of structural changes to the arterial wall (e.g., increased collagen deposition and elastin fragmentation) as well as functional changes that affect arterial tone, including increases in blood pressure and sympathetic nerve activity 24 and impaired endothelial function. These data suggest that NR lowers aortic stiffness in humans through a mechanism that is not entirely dependent on the reduction in blood pressure, as only ˜10% of the variance in the reduction in PWV was explained by the reduction in systolic blood pressure. Likewise, NR lowered PWV by a similar degree in all subjects, regardless of baseline blood pressure status. Supplementation with the alternative NAD+ boosting compound, nicotinamide mononucleotide (NMN), reverses aortic stiffening in old mice to youthful levels by reversing age-related increases in collagen deposition and elastic fragmentation (de Picciotto, N. E. et al. Nicotinamide mononucleotide supplementation reverses vascular dysfunction and oxidative stress with aging in mice. Aging Cell 15, 522-530 (2016)). Likewise, activation of SIRT-1 with the pharmacological compound SRT1720 prevented the development of arterial stiffness in klotho deficient mice through a mechanism involving reduced collagen deposition and elastin fragmentation. NR may have reduced arterial stiffness in the present study through similar alterations to the intrinsic mechanical properties of the aorta.

### NR Supplementation and Endothelin-1

Endothelin-1 (ET-1) is a 21-amino-acid peptide involved in the regulation of vascular tone that becomes activated with aging and contributes to risk of hypertension and atherosclerosis (Stauffer et al. Endothelin-1, aging and hypertension. 23, 350-355 (2008)). ET-1 is synthesized the vascular endothelium and is secreted into the surrounding vascular smooth muscle cells where it acts as a vasoconstrictor through its binding to specific ET receptors. The secretion of ET-1 by endothelial cells is suppressed by resveratrol, a polyphenol compound found in red wine that has been shown to activate SIRT-1 (Nicholson et al. Physiological concentrations of dietary polyphenols regulate vascular endothelial cell expression of genes important in cardiovascular health. Br. J. Nutr. 103, 1398-403 (2010)). Supplementation with resveratrol decreases circulating ET-1 levels in animal models of hypercholesterolemia and cardiac hypertrophy, suggesting that activation of SIRT-1 may lower the risk of cardiovascular diseases through its effects on ET-1 signaling (Zou, J. G. et al. Effect of red wine and wine polyphenol resveratrol on endothelial function in hypercholesterolemic rabbits. Int. J. Mol. Med. 11, 317-320 (2003); Liu, Z. et al. Effects of trans-resveratrol on hypertension-induced cardiac hypertrophy using the partially nephrectomized rat model. Clin. Exp. Pharmacol. Physiol. 32, 1049-1054 (2005)). NAD+ is the critical co-substrate for SIRT-1 activation; thus, supplementation with NAD+ boosting compounds such as nicotinamide riboside may reduce blood pressure through a SIRT-1-mediated suppression of ET-1 signaling.

Although the foregoing invention has been described in some detail by way of illustration and example for purposes of clarity of understanding, the descriptions and examples should not be construed as limiting the scope of the invention.

1-36. (canceled)

37. A method for treating elevated/stage 1 hypertension (pre-hypertension) in a subject in need of treatment thereof, comprising administering to the subject a therapeutically effective amount of at least one nicotinamide adenine dinucleotide (NAD+) increasing compound, or a pharmaceutically acceptable salt thereof.

38. The method of claim 37, wherein the nicotinamide adenine dinucleotide (NAD+) increasing compound is selected from nicotinamide riboside, a functional homolog thereof, a prodrug thereof, and a pharmaceutically acceptable salt thereof.

39. The method of claim 37, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

40. A method for treating stage 2 or above hypertension in a subject in need of treatment thereof, comprising administering to the subject a therapeutically effective amount of at least one nicotinamide adenine dinucleotide (NAD+) increasing compound, or a pharmaceutically acceptable salt thereof.

41. The method of claim 40, wherein the nicotinamide adenine dinucleotide (NAD+) increasing compound is selected from nicotinamide riboside, a functional homolog thereof, a prodrug thereof, and a pharmaceutically acceptable salt thereof.

42. The method of claim 40, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

43. A method of lowering blood pressure in a subject, comprising administering to the subject a therapeutically effective amount of at least one nicotinamide adenine dinucleotide (NAD+) increasing compound, or a pharmaceutically acceptable salt thereof.

44. The method of claim 43, wherein the nicotinamide adenine dinucleotide (NAD+) increasing compound is selected from nicotinamide riboside, a functional homolog thereof, a prodrug thereof, and a pharmaceutically acceptable salt thereof.

45. The method of claim 43, wherein the subject has a systolic blood pressure in a range of 120 mmHg to 139 mmHg, a diastolic blood pressure in the range of 80 mmHg to 89 mmHg or a combination of a systolic blood pressure in a range of 120 mmHg to 139 mmHg and a diastolic blood pressure in the range of 80 mmHg to 89 mmHg.

46. The method of claim 43, wherein the subject has a systolic blood pressure of at least 140 mmHg, a diastolic blood pressure of at least 90 mmHg, a mean arterial pressure of at least 106 mmHg or a combination of a systolic blood pressure of at least 140 mmHg and a diastolic blood pressure of at least 90 mmHg.

47. The method of claim 43, wherein the administration of the at least one compound lowers the systolic blood pressure, diastolic blood pressure, mean arterial pressure or a combination of the systolic blood pressure and diastolic blood pressure of the subject.

48. A method for preventing or reducing arterial stiffness in a subject in need of treatment thereof, comprising administering to the subject a therapeutically effective amount of at least one nicotinamide adenine dinucleotide (NAD+) increasing compound, or a pharmaceutically acceptable salt thereof.

49. The method of claim 48, wherein the nicotinamide adenine dinucleotide (NAD+) increasing compound is selected from nicotinamide riboside, a functional homolog thereof, a prodrug thereof, and a pharmaceutically acceptable salt thereof.

50. The method of claim 48, wherein the arterial stiffness is measured in the subject through carotid-femoral pulse wave velocity, an index of aortic stiffness assessed using arterial tonometry, or Doppler ultrasound.

51. The method of claim 48 further comprising administering to the subject a therapeutically effective amount of at least one antihypertensive compound with the at least one nicotinamide adenine dinucleotide (NAD+) increasing compound, or a pharmaceutically acceptable salt thereof.

52. The method of claim 48 further comprising administering to the subject a therapeutically effective amount of at least one compounds selected from a diuretic, an angiotensin converting enzyme (ACE) inhibitor, an angiotensin II receptor blocker (ARB), a beta-blocker, a calcium-channel blocker, a statin, an organic nitrate, and an organic nitrite.
